(19) United States
(12) Patent Application Publication
(10) Pub. No.: US 2026/0334872 A1
(43) Pub. Date: Oct. 29, 2026

(54) INTELLIGENT INFUSION BASED ON ANTICIPATING PROCEDURAL EVENTS

(71) Applicant: CareFusion 303, Inc., San Diego, CA (US)

(72) Inventors: Daniel M. ABAL, San Diego, CA (US); Brendan Burgess, Poway, CA (US)

(21) Appl. No.: 19/119,665

(22) PCT Filed: Oct. 11, 2022
(86) PCT No.: PCT/US2022/046316

§ 371 Date: Apr. 9, 2025

(51) Int. Cl. A61B 5/00

(52) U.S. Cl. CPC A61B 5/4839; A61B 5/7267; A61B 5/7275; A61B 5/7282; A61B 5/7435

(57) ABSTRACT

An intelligent control device provides an option to select a medical procedure and receives a selection of the medical procedure. The intelligent control device also operates an infusion device to administer a medication at a first rate. Further, the intelligent control device identifies a set of events associated with the selected medical procedure, including a procedural event expected to cause a physiological change for which an administration of the medication is prescribed. Moreover, the intelligent control device determines that the procedural event is about to occur based on the infusion information, the physiological information, or the selected medical procedure. After determining that the procedural event is about to occur, and before the procedural event occurs, the intelligent control device prompts an operator to cause an adjustment of the infusion device to administer the medication at a second rate; or the intelligent control device causes the adjustment of the infusion device.

### BACKGROUND

Biometric sensors are often used to monitor the condition of a patient during a controlled infusion. The information provided by biometric sensors allows a clinician to respond to physiological changes in the patient, thereby maintaining better control of the infusion therapy. If a sensor indicates, for example, that the patient is in pain, an anesthesiologist can respond by adjusting an amount of an analgesic drug provided to the patient by an infusion pump. Similarly, if a sensor indicates that a patient is prematurely exiting anesthesia, the anesthesiologist may respond by providing an anesthetic drug to the patient.

At times, however, changes in biometric signals may lead to overcorrection in medication delivery. Overcorrection can lead to overmedication, which may result in harm to the patient or wasted medical resources. Additionally, correction often occurs after the physiological metric has already exited a target safety zone, again risking harm to the patient. Accordingly, there is a need for an alternative approach to the administration of infusions that incorporate physiological feedback.

### SUMMARY

According to various aspects of the subject technology, an intelligent control device for intelligent infusion includes a control interface configured to exchange infusion information with an infusion device and physiological information with a patient sensor. The intelligent control device also includes a processor and a non-transitory, computer-readable medium storing instructions that, when executed by the processor, cause the intelligent control device to provide, via a display device, a user interface comprising an option to select a medical procedure. Further, the instructions cause the intelligent control device to receive, via the user interface, a selection of the medical procedure. Moreover, the instructions cause the intelligent control device to operate, via the control interface, the infusion device to administer a medication at a first rate. Additionally, the instructions cause the intelligent control device to identify a set of events associated with the selected medical procedure, wherein the set of events comprises a procedural event that is expected to cause a physiological change for which an administration of the medication is prescribed. Also, the instructions cause the intelligent control device to determine that the procedural event is about to occur based on the infusion information, the physiological information, or the selected medical procedure. Moreover, the instructions cause the intelligent control device, responsive to determining that the procedural event is about to occur, and prior to an occurrence of the procedural event: (a) prompt, via the display device, an operator of the infusion device to cause an adjustment of the infusion device to administer the medication at a second rate different from the first rate; or (b) cause, via the control interface, the adjustment of the infusion device to administer the medication at the second rate.

According to various aspects of the subject technology, a computer-implemented method of intelligent infusion includes activating an intelligent control device comprising a control interface configured to exchange infusion information with an infusion device and physiological information with a patient sensor. Additionally, the computer-implemented method includes providing, via a display device, a user interface comprising an option to select a medical procedure. Further, the computer-implemented method includes receiving, via the user interface, a selection of the medical procedure. Moreover, the computer-implemented method includes operating, via the control interface, the infusion device to administer a medication at a first rate. Also, the computer-implemented method includes identifying a set of events associated with the selected medical procedure, wherein the set of events comprises a procedural event that is expected to cause a physiological change for which an administration of the medication is prescribed. Moreover, the computer-implemented method includes determining that the procedural event is about to occur based on the infusion information, the physiological information, or the selected medical procedure. Furthermore, the computer-implemented method includes, responsive to determining that the procedural event is about to occur, and prior to an occurrence of the procedural event: (a) prompting, via the display device, an operator of the infusion device to cause an adjustment of the infusion device to administer the medication at a second rate different from the first rate; or (b) causing, via the control interface, the adjustment of the infusion device to administer the medication at the second rate.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Detailed Description below, in conjunction with the Figures. Like reference numerals refer to corresponding parts throughout the Figures and Description.

FIG. 1 depicts an example of an institutional patient care system of a healthcare organization, according to aspects of the subject technology.

FIG. 2 is a conceptual diagram illustrating an example system architecture, including an example resource management unit, according to aspects of the subject technology.

FIG. 3 is an example timeline for a knee replacement surgery, according to aspects of the subject technology.

FIGS. 4A and 4B are example line plots that each include a biometric sensor readout and a medication infusion metric, according to aspects of the subject technology.

FIG. 5 is an example user interface including a prompt to initiate administration of a medication in anticipation of a physiological change for which the medication is prescribed, according to aspects of the subject technology.

FIG. 6 depicts an example system for detection of surgical events, according to aspects of the subject technology.

FIG. 7 illustrates a training flow for a machine learning model in conjunction with a closed-loop algorithm, according to aspects of the subject technology.

FIG. 8 depicts an example process for intelligent infusion, according to aspects of the subject technology.

FIG. 9 is a conceptual diagram illustrating an example electronic system for intelligently determining when to initiate an infusion of a medication, according to aspects of the subject technology.

### DETAILED DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In some instances, well-known methods, procedures, components, circuits, and networks have not been described in detail so as not to unnecessarily obscure aspects of the implementations.

There is a need for an alternative approach to incorporating physiological feedback in infusions of medication. According to various implementations, the subject technology described herein provides an intelligent control device that can source physiological data in order to determine when a significant change, or a “spike” (e.g., an upward spike, a downward spike), in a sensor signal is about to occur. After predicting the potential spike in the sensor signal, the intelligent control device can cause an infusion device to adjust (e.g., increase, decrease) a flow rate of a medication in order to prevent or at least decrease the severity of the spike and maintain the physiological condition of the patient. This is especially valuable for medical procedures that require physiological metrics of the patient to remain in a “safety zone” based on, for example, bi-spectral index (BIS) values or electromyography (EMG) sensor readings while a patient is under anesthesia.

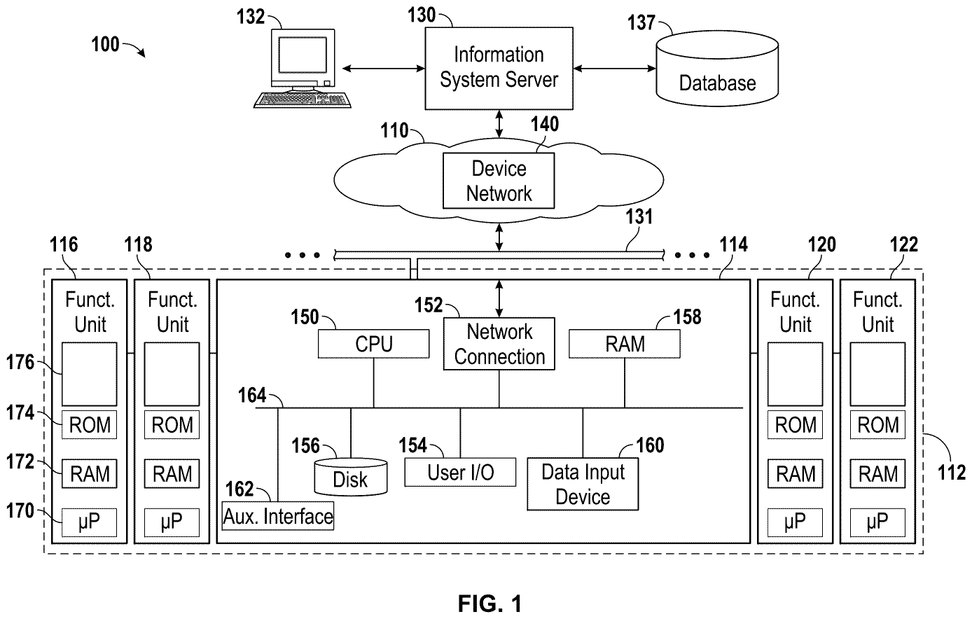

FIG. 1 depicts an example of an institutional patient care system 100 of a healthcare organization, according to aspects of the subject technology. In FIG. 1, a patient care device (“PCD”) 112 is connected to an internal healthcare network 110. The PCD 112 may include various ancillary medical devices such as an infusion pump, a vital-signs monitor, a medication dispensing device (e.g., a cabinet, a tote), a medication preparation device, an automated dispensing device, a module coupled with one of the aforementioned devices (e.g., a syringe pump module coupled with an infusion pump), or other similar devices. Each element of PCD 112 is connected to an internal healthcare network 110 by a transmission channel 131. Transmission channel 131 may be a wired or wireless transmission channel (e.g., an 802.11 wireless local area network (LAN)).

In some implementations, internal healthcare network 110 also includes computer systems located in various departments throughout a hospital. For example, internal healthcare network 110 of FIG. 1 optionally includes computer systems associated with an admissions department, a billing department, a biomedical engineering department, a clinical laboratory, a central supply department, one or more unit station computers or a medical decision support system. As described further below, internal healthcare network 110 may include discrete subnetworks. In the depicted example, internal healthcare network 110 includes a device network 140 by which PCD 112 (and other devices) may communicate in accordance with normal operations.

Additionally, institutional patient care system 100 may incorporate a separate information system server 130, the function of which will be described in more detail below. Moreover, although the information system server 130 is shown as a separate server, the functions and programming of the information system server 130 may be incorporated into another computer, if such is desired by engineers designing the institution's information system. Institutional patient care system 100 may further include one or multiple device terminals 132 for connecting and communicating with information system server 130. Device terminals 132 may include personal computers, personal data assistants, mobile devices (e.g., laptops, tablet computers, augmented reality devices, smartphones) configured with software for communicating with information system server 130 via internal healthcare network 110.

PCD 112 comprises a system for providing patient care, such as that described in U.S. Pat. No. 5,713,856 to Eggers et al., which is incorporated herein by reference for that purpose. PCD 112 may also include or incorporate pumps, physiological monitors (e.g., a heart rate (HR) monitor, a blood pressure monitor, an ECG, an EEG, a pulse oximeter), therapy devices, or other drug delivery devices according to the teachings set forth herein. In the depicted example, PCD 112 comprises a main frame infusion controller 114, connected to one or more functional modules 116, 118, 120, and 122. Main frame infusion controller 114 includes a central processing unit (CPU) 150 connected to a memory (e.g., random access memory (RAM) 158) and one or more interface devices such as user interface device 154, a coded data input device 160, a network connection 152, and an auxiliary interface 162 for communicating with additional modules or devices. Additionally, in some implementations, main frame infusion controller 114 includes a main, non-volatile storage unit 156 (e.g., a hard disk drive, a non-volatile flash memory) for storing software and data. Further, in some implementations, main frame infusion controller 114 includes one or more internal buses 164 for interconnecting the aforementioned elements.

In various implementations, user interface device 154 includes a touch screen for displaying information to a user and allowing a user to input information by touching defined areas of the screen. Additionally, or alternatively, user interface device 154 could include means (specifically configured with one or more features described) for displaying and inputting information, such as a monitor, a printer, a keyboard, softkeys, a mouse, a track ball and/or a light pen.

Data input device 160 may be a bar code reader capable of scanning and interpreting data printed in bar coded format. Additionally, or in the alternative, data input device 160 can be a specifically-configured device for entering coded data into a computer, such as a device(s) for reading a magnetic strip, radio-frequency identification (RFID) devices whereby digital data encoded in RFID tags or smart labels (defined below) are captured by the reader 160 via radio waves, Personal Computer Memory Card International Association (PCMCIA) smart cards, radio frequency cards, memory sticks, CDs, DVDs, or other analog or digital storage media. Other examples of data input device 160 include a voice activation or recognition device or a portable personal data assistant (PDA). Depending upon the types of interface devices used, user interface device 154 and data input device 160 may be the same device.

Although data input device 160 is shown in FIG. 1 to be disposed within main frame infusion controller 114, it is recognized that data input device 160 may be integral within pharmacy system 134 or located externally and communicating with pharmacy system 134 through an RS-232 serial interface or other appropriate communication means.

Auxiliary interface 162 may be an RS-232 communications interface. However, other means for communicating with a peripheral device in the described environments, such as a printer, patient monitor, infusion pump or other medical device, may be used without departing from the subject technology. Additionally, data input device 160 may be a separate functional module, such as modules 116, 118, 120, and 122, and configured to communicate with main frame infusion controller 114, or other systems on the network, using suitable programming and communication protocols.

Network connection 152 may be a wired or wireless connection, such as by Ethernet, WiFi, BLUETOOTH, an integrated services digital network (ISDN) connection, a digital subscriber line (DSL) modem, or a cable modem. Direct or indirect network connection may be used, including, but not limited to a telephone modem, an MIB system, an RS232 interface, an auxiliary interface, an optical link, an infrared link, a radio frequency link, a microwave link, a personal area network connection, a local area network connection, a cellular link, or a WLANS connection or other wireless connection.

Functional modules 116, 118, 120, and 122 are specially configured devices for providing care to a patient or for monitoring patient condition. At least one of functional modules 116, 118, 120, and 122 may be an infusion pump module such as an intravenous infusion pump for delivering medication or other fluid to a patient. For the purposes of this discussion, functional module 116 is an infusion pump module. Each of functional modules 118, 120, and 122 may be a patient treatment or monitoring device including, but not limited to, an infusion pump, a syringe pump, a patient-controlled analgesia (PCA) pump, an epidural pump, an enteral pump, a blood pressure monitor, a pulse oximeter, an EKG monitor, an EEG monitor, an HR monitor, an intracranial pressure monitor, or the like. Functional modules 118, 120, and/or 122 may also be a printer, scanner, bar code reader, or other peripheral input, output, or input-output device.

Each of functional modules 116, 118, 120, and 122 communicates directly or indirectly with main frame infusion controller 114, with main frame infusion controller 114 providing overall monitoring and control of PCD 112. Functional modules 116, 118, 120, and 122 may be connected physically and electronically in serial fashion to one or both ends of main frame infusion controller 114 as shown in FIG. 1, or as detailed in Eggers et al. However, it is recognized that there are other means for connecting functional modules with the main frame infusion controller 114 that may be utilized without departing from the subject technology. It will also be appreciated that devices such as pumps or patient monitoring devices that provide sufficient programmability and connectivity may be capable of operating as stand-alone devices and may communicate directly with the network without being connected through a separate interface or control unit. As described above, additional medical devices or peripheral devices may be connected to PCD 112 through one or more auxiliary interfaces 162. Each of functional modules 116, 118, 120, and 122 may also include module-specific components 176, a microprocessor 170, a volatile memory 172, and a nonvolatile memory 174 for storing information.

It should be noted that while four functional modules are shown in FIG. 1, any number of devices may be connected directly or indirectly to main frame infusion controller 114. The number and type of functional modules described herein are intended to be illustrative, and in no way limit the scope of the subject technology. Module-specific components 176 include specifically configured components necessary for operation of a particular module, such as a pumping mechanism for infusion pump module 116.

While each functional module may be capable of a least some level of independent operation, main frame infusion controller 114 monitors and controls overall operation of PCD 112. For example, as will be described in more detail below, main frame infusion controller 114 provides programming instructions to the functional modules 116, 118, 120, and 122 and monitors the status of each module.

PCD 112 is capable of operating in several different modes, or “personalities,” with each personality defined by a configuration database. The configuration database may be a non-volatile storage unit 156 internal to PCD 112, or it may be an external database 137. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics, or psychological characteristics. As used herein, patient-specific information also includes care provider information (e.g., a physician identification) or the location of a PCD 112 in a hospital or hospital computer network (e.g., internal healthcare network 110). Patient care information may be entered through network connection 152, user interface device 154, data input device 160 or auxiliary interface 162, and it may originate from anywhere in internal healthcare network 110, such as, for example, from a pharmacy server, admissions server, laboratory server, and the like.

Medical devices incorporating aspects of the subject technology may be equipped with a network interface module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, PCD 112 and internal healthcare network 110 may communicate via automated interaction, manual interaction, or a combination of the two. Automated interaction may be continuous or intermittent and may occur through direct network connection 154 (as shown in FIG. 1), or through RS232 links, MIB systems, RF links such as Bluetooth, IR links, PANS, LANS, WLANS, digital cable systems, telephone modems, or other wired or wireless communication means.

Manual interaction between PCD 112 and internal healthcare network 110 involves physically transferring, intermittently or periodically, data between the systems using, for example, user interface device 154, coded data input device 160, bar codes, computer disks, portable data assistants, memory cards, or other media for storing data. The communication means in various aspects is bidirectional with access to data from as many points of the distributed data sources as possible. Decision-making can occur at a variety of places within internal healthcare network 110. For example, and not by way of limitation, decisions can be made in information system server 130, decision support, a remote data server, hospital department or unit stations, or within PCD 112 itself.

All direct communications with medical devices operating on a network in accordance with the subject technology may be performed through information system server 130, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication.

In some implementations, functional modules 116, 118, 120, and 122 include plug-in ports for expansion. Accordingly, an external medication delivery module may be attached to PCD 112 by coupling a connector through a plug-in port (e.g., an electrical terminal) so that the external medication delivery module may transmit and receive information to and from main frame infusion controller 114. In some implementations, the added external medication delivery module may also receive power from main frame infusion controller 114 through a plug-in port.

Main frame infusion controller 114 may include a main display, a memory, and a processor. Additionally, main frame infusion controller 114 may be configured to display operational parameters and medication delivery status, and further information associated with each of functional modules 116, 118, 120, and 122. According to various implementations, module displays may also display physiological data (e.g., vital signs) associated with a patient.

A main display (e.g., user interface 154) may be configured to display one or more user interfaces for the display of operational parameters or other data associated with a functional module 116, 118, 120, and 122, and/or physiological properties associated with the patient. The main display may include multiple user interfaces, with each individual user interface graphically displaying information for a respective one of functional modules 116, 118, 120, and 122, including information that may also be displayed on corresponding module displays. In some implementations, main frame infusion controller 114 includes a communications module (e.g., an antenna), configured to communicate wirelessly with a controller or with a network.

When a functional module 116, 118, 120, and 122 initiates an infusion of a medication to a patient, the main frame infusion controller 114 is configured to create and manage an infusion session within a memory of the control module (or related module). For the purpose of this disclosure, the infusion session includes state information of the PCD 112, its main frame infusion controller 114, and/or its associated modules, which is recorded and saved to memory during a particular period of time. The state information includes, but is not limited to, records of parameter values utilized by the PCD 112, its control module, and/or its associated modules during the period of time, and/or records physiological data collected during the period of time. During the infusion, physiological data associated with the patient is recorded within the session. Operating parameter values, as well as modifications to the operating parameters of the PCD 112, its control module, and/or modules are also recorded in the session.

If not already logged into the PCD 112, a clinician may scan their badge proximate to a sensor (e.g., user interface 154, data input device 160) on the PCD 112, and the PCD 112 may attempt to authenticate the clinician by sending the clinician's scanned identification to the information system server 130. The clinician's badge may incorporate a radio frequency identification device (RFID), which is read by a scanner integrated with the PCD 112 or a portable scanner associated with the PCD 112.

The clinician may scan their badge at the main frame infusion controller 114 to identify and authorize the clinician to initiate the administration of a medication. Once the clinician is associated with the PCD 112 and/or functional module(s) 116, 118, 120, 122, the clinician's identification is associated with the session. The same is applicable with a patient. The clinician may scan a patient's wristband with a portable scanner or use the sensor on the PCD 112 (or its control module) to associate the patient with the PCD 112 and/or module(s) 116, 118, 120, 122 (and a session).

In some implementations, main frame infusion controller 114 of PCD 112 is configured to generate a graphical representation of the infusion session, and display (e.g., in a display) the graphical representation, including a graphical visualization of parameters of the infusion during the session and modifications to the parameters, together with physiological data obtained during the session. The graphical representation may include pseudo-identifiers for unknown data until such data is substituted with known identifiers. At that time, the graphical representation is displayed with the known patient identifiers.

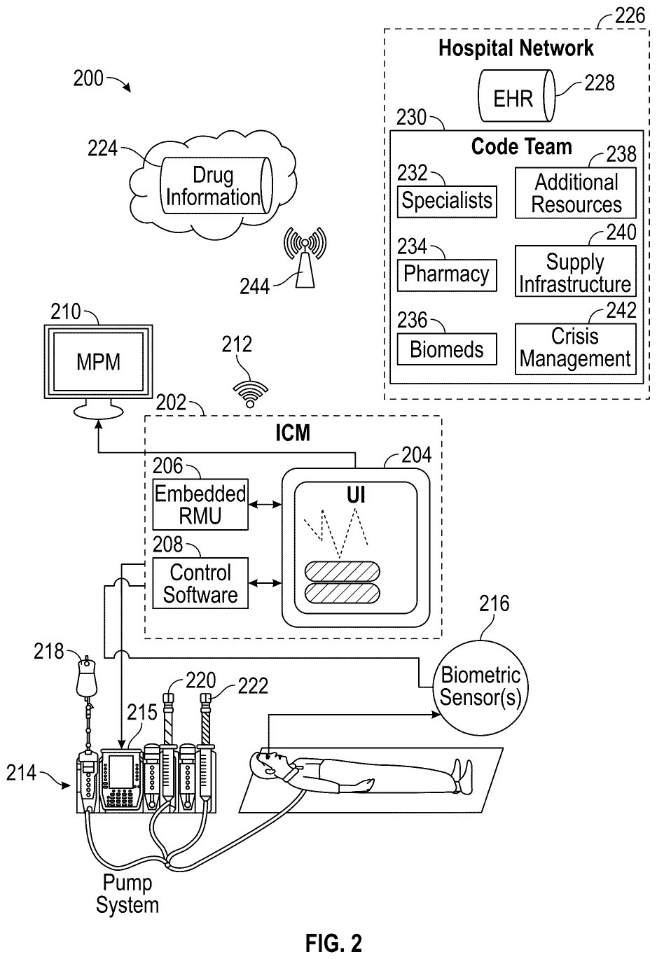

FIG. 2 is a conceptual diagram illustrating an example system architecture, including an example resource management unit, according to aspects of the subject technology. In the depicted example, a care provision system 200 includes an intelligent control module (“ICM”) 202. The ICM 202 (or infusion device control system) provides direct control over connected medical devices to assist the clinician in providing a more centralized control and management of the devices.

The ICM 202 provides a modular interface system by which various biometric sensors 216 used in a medical environment may be connected in a generic way to facilitate control over the connected medical devices. For example, the biometric sensors 216 may include an HR monitor, an oxygen sensor, and an intravenous (IV) flow rate monitor, all of which may be connected to the ICM 202 to facilitate, in addition to input by the clinician, centralized control of one or more infusion devices. The biometric sensors 216 may also include a BIS sensor 216 to assess the depth of anesthesia during an operation, as will be discussed in further detail below.

According to various implementations, the ICM 202 is a standalone device, separate from the infusion device(s). In some implementations, the ICM 202 is a mobile device with a form factor like a tablet computer. The ICM 202 may be portable with a rechargeable power source (e.g., a battery). In some implementations, the ICM 202 may be configured to integrate with (e.g., share power with) an infusion device 214. The ICM 202 may be further configured to connect to a server or cloud-based system for data input, data coordination, and reporting purposes. Examples of cloud-based systems include a cloud-based drug information database 224 (or formulary), and a hospital network 226 (e.g., internal healthcare network 110) that includes an electronic medical record (EMR) system or database 228.

The hospital network 226 may also include a code team 230 system and/or network that responds to code alarms. The code team 230 includes specialists 232 (human or AI), a pharmacy 234, biomedical technicians 236 (human or AI), crisis management resources 242, supply infrastructure 240, and additional resources 238 for responding to requests made to the code team 230.

In the depicted example, the ICM 202 includes a control unit 208, including one or more processors and/or control software and/or control algorithms. In some implementations, control unit 208 includes connectivity circuitry. In some implementations the control unit 208 includes software to enable closed-or semi-closed-loop control capabilities for one or medical devices at a point of use. In some implementations, the ICM 202 provides an external interface, for example a user interface 204, for interaction between an IV infusion device 214 and one or more different physiological or biometric sensors 216, and for providing input parameters that may be used to control the titration of IV infusions of medications to a patient.

In some implementations, the closed-loop system provides control for the IV infusion of the medication by an infusion device 214 according to the feedback provided by the biometric sensors 216 used to monitor the therapy being performed. In situations where the IV medication is not being delivered as a result of abnormal conditions such as the IV line being occluded, leaking, or not being connected to the patient, the sensors 216 may indicate that the patient is not responding to the therapy. The subject technology provides feedback (e.g., a notification, an alarm) to the clinician to alert the clinician of a possible fault condition that should be addressed, in some instances before a critical situation occurs.

The ICM 202 can incorporate control software (e.g., one or more algorithms in the control unit 208) that can be tailored to specific or general medical treatments. The ICM 202 may receive infusion status information from infusion device 214. The infusion status information may include, for example, an identification of the medication, a flow rate of an administration of medication to a patient by the infusion device, a VTBI (volume to be infused), delivery duration, upstream or downstream pressure, and the like. While the infusion device 214 may have its own safety system, the ICM 202 may be preprogrammed to determine safety events such as events specific to a particular procedure. The ICM 202 may determine, based on sensor data from sensor(s) 216 and infusion status information, that a safety event is likely to occur within a predetermined period of time (e.g., programmed into the ICM or determined by AI based on training models for the procedure being undertaken).

A closed-loop control system as described herein generally refers to a system that does not rely on external manual inputs to deliver a therapy. Once configured, the closed-loop system can autonomously provide a therapy, receive feedback from one or more sensors 216, and, based on the feedback, automatically adjust the therapy as needed. For example, the ICM 202 may determine, during an administration of a medication, an expected trend in a physiological property during a predetermined time period based on sensor data for a prior period of time, a dose of the medication provided to the patient, and the one or more physical parameters of the patient. The ICM 202 may then cause the infusion device to adjust the dose of the medication to cause the physiological property to follow an expected trend within a predetermined time period.

Having the ICM 202 as a separate unit from the medical device provides several advantages. For example, the advancement of sensors (e.g., biometric sensors 216) may be much more rapid than the development of infusion pump systems (e.g., pump device 214). Accordingly, the control system may accommodate for these changes more quickly. Further, it may be desirable to update control algorithms to address changes in treatment methods, available medications, and patient physiology. For this reason, it may be desirable to have the algorithm reside in a component different than the pump system, in order to accommodate more frequent changes.

Moreover, machine learning and artificial intelligence may account for patient variations related to physiological properties such as age, genetics, health history, and other characteristic and environmental factors. Systems incorporating such capabilities may involve large databases and complex programs requiring powerful microprocessors and data storage capabilities to perform the timely and accurate computation needed. These systems are generally not capable of running on the systems currently available with the IV pumps alone, but may reside on the information system server 130 or other cloud-based system accessible from the ICM 202.

Additionally, the ICM 202 of the subject technology may be adaptable to a variety of pump systems and sensor inputs. The ICM 202 may be configured to add wireless, Bluetooth and LAN connections to pump systems that do not currently have it available. For example, FIG. 2 shows the ICM 202 having a wireless connection 212 for communication with a network system 244 at the hospital. Adding such communications to the infusion device 214 may enable other capabilities such as remote monitoring and control of the infusion pumps, and facilitating access to EMR 228. Accordingly, by integrating the electrical and processing components separate from the pump, the subject technology facilitates integration of additional capabilities without needing to modify the pump's housing and electronics. Separating the physiological sensing and control systems from the infusion pump system may further provide for a more streamlined regulatory approval process.

According to various implementations, the control software of control unit 208 is separate from the embedded firmware of the pumps and the sensors, which can facilitate a scalable and rapidly configured system to provide closed-loop control of medical treatments. Moreover, the ICM 202 can be configured to operate with a multitude of sensors by way of electrical connectors and/or wireless communication. The ICM 202 may include one or more microprocessors and algorithms to provide signal conditioning and/or conversion of the sensor signals to the appropriate physiological properties for the connected medical device. The parameters may then be used in control algorithms to provide control to, for example, an infusion pump to deliver the necessary medications or fluids for a desired clinical outcome. As used herein, “connecting” devices or “operably connecting” devices may include establishing a physical (e.g., wired) or virtual (e.g., wireless) connection between the devices.

The user interface 204 of ICM 202 can include a display module or a touch-sensitive display module configured to provide a user interface for display of information pertaining to patient physiological status, as well as system control status. In some implementations, the user interface 204 includes circuitry within the ICM 202 housing that provides display information to an external display device. An example of such an external display device includes a multi-parameter monitor (MPM) 210. The MPM 210 may, for example, display various vital statistics (e.g., electrocardiogram saturation level (SpO2)) of a patient in a surgery room such that the vital statistics are visible to the clinicians (e.g., all the clinicians) in the room. The display on the ICM 202 may be mirrored via an associated MPM 210 to provide information to devices connected to the MPM 210 and/or clinicians involved in the treatment.

The ability of the ICM 202 to connect to another display (e.g., the MPM 210) provides modular scalability. For example, a more extensive user interface may be beneficial in some use cases. The more extensive user interface may include displays of data and graphs, for which a larger high-resolution display may be used. Some use cases may benefit from a display having minimal information and a corresponding user interface to support such a configuration. A smaller, space saving and/or lower-cost, locally connected display may then be used.

According to various implementations, the ICM 202 includes a resource management unit (“RMU”) 206. The RMU 206 stores protocol information (e.g., information about processes and steps associated with therapies and patients), making the information readily accessible in the ICM 202. In some implementations, the RMU 206 provides information to the user interface 204, allowing the system to more efficiently (e.g., using fewer device resources such as memory, power, processing cycles, user interface area, etc.) present and navigate through pertinent information (e.g., using touch inputs on a touch screen). According to variously implementations, the RMU 206 guides the user to the order of steps and information needed at the appropriate time thereby avoiding expenditures of resources on information that is untimely or irrelevant to the currently detected condition.

In some implementations, the ICM 202 may access additional resources through the communication capabilities of the RMU 206. For example, the RMU 206 may access additional resources relating to medication information or dosage calculations from an internal or remote storage (e.g., from drug information database 224). In some implementations, the RMU 206 can cause the user interface 204, and/or other operably connected user devices to display a dosage calculator for a patient when a particular medication is to be administered. In some implementations, the dosage calculator is a weight-based calculator that accounts for a weight of the patient (e.g., the weight of the patient is provided to the ICM 202 by manual entry by a clinician, or based on information received from the EMR database 228). In some implementations, calculations for weight-based medications can be readily accessed through the user interface 204 or the one or more user devices communicatively connected to the ICM 202.

A patient's electronic health record may also be readily accessed directly through the ICM 202 to provide critical health and allergy information to the clinicians. The additional resources relating to medication information accessed by the ICM 202 via the RMU 206 may include information about a compatibility of medications that a patient is prescribed to receive. In some implementations, the RMU 206 can cause the user interface 204, and/or other user devices to display mixing ratio instructions. For example, the RMU 206 may cause the user interface 204 to specify a current or programmed flow rate of the first fluid from the syringe pump 220, a current or programmed flow rate of the second fluid from the syringe pump 222, and a current or programmed flow rate of the infusion fluid from the infusion bag 218 to facilitate an appropriate mixture for administration to the patient during the infusion.

In one example, the RMU 206 may access additional resources by allowing a clinician to electronically deliver or make pharmacy requests via the ICM 202 to the pharmacy 234. The RMU 206 can cause the user interface 204 to display controls that allow a clinician to summon one or more specialists for consultation (e.g., virtually using cameras communicatively connected to the ICM 202 and/or data from the biometric sensors 216; or request for an in-person consultation at the patient's location).

In some implementations, the RMU 206 may connect the clinician to an AI. The RMU 206 can cause the user interface 204 to display controls that allow a clinician to send code alarms so that additional resources and personnel (e.g., human or AI) can respond to a medical issue. The RMU 206 can cause the user interface 204 to display controls for equipment requests or for other resources needed by the patient. Thus, in the cases where a hospital code would need to be issued, a clinician can simply use the RMU 206 to bring up checklists that would then present appropriate codes available for specific requests to be sent via the hospital network 244. In some implementations, the user interface may include a control element that, when activated, transmits a control message to a device in communication with the RMU 206 to request a resource.

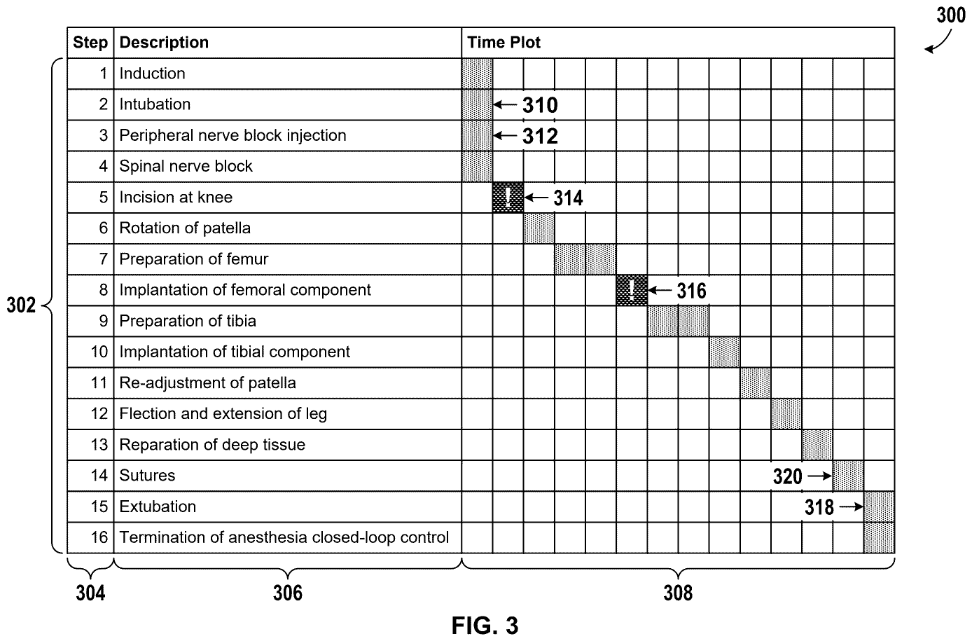

FIG. 3 is an example timeline 300 for a knee replacement surgery, according to aspects of the subject technology. As illustrated, the timeline 300 includes events 302, as well as corresponding step numbers 304, descriptions 306, and a time plot 308. Each of the events 302 represents an event that is expected to occur in the knee replacement surgery. The information contained within the timeline 300 may be stored in the main frame infusion controller 114 (e.g., in non-volatile storage unit 156 and/or RAM 158), in the ICM 202, in the external database 137, or in another electronic device or database. According to various implementations, the ICM 202 retrieves the information contained within the timeline 300 (e.g., from memory and/or a database) to determine the timing of an event 302.

The descriptions 306 offer a brief description for each of the events 302. For example, the first event (i.e., step 1) of the knee replacement surgery includes induction; the second (i.e., step 2), intubation; the third (i.e., step 3), a peripheral nerve block injection; and so on. As for when the events 302 are expected to occur, the step numbers 304 (e.g., 1, 2, 3, etc.) offer a general indication of chronology. Generally, low-numbered events precede high-numbered events (e.g., induction precedes rotation of the patella); though, some similarly-numbered events may occur simultaneously. In some implementations, the events 302 may not be sequentially ordered.

The time plot 308 includes more detailed information regarding when the events 302 are expected to occur with respect to other events in the timeline 302, including which events are expected to overlap, occur sequentially, or occur simultaneously (or relatively simultaneously). The time plot 308 flows from left to right, with the leftmost column corresponding to the beginning of the knee replacement surgery and the rightmost column corresponding to the end of the surgery. Each row in the time plot 308 corresponds to one of the events 302. Accordingly, a shaded cell in a particular column and row indicates at what time (i.e., based on the column) a particular event (i.e., based on the row) is expected to occur. For example, the preparation of the tibia event (i.e., step 9) is expected to occur in the middle of the surgery because the shaded cell in the row of the event falls in a middle column of the time plot 308.

For example, the timeline 300 indicates that intubation is expected to occur before an incision is made at the patient's knee, and that extubation is expected to occur after sutures are applied, because the shaded cell 310 corresponding to intubation precedes the shaded cell 314 corresponding to the incision, and the shaded cell 318 corresponding to extubation follows the shaded cell 320 corresponding to the application of sutures. As another example, the timeline 300 indicates that intubation and a peripheral nerve block injection are to occur relatively simultaneously (e.g., within five, ten, etc. minutes of each other), as respective corresponding shaded cells 310 and 312 are in the same column of the time plot 308.

Furthermore, the time plot 308 includes information regarding the duration of each of the events 302. Most of the events 302 are associated with only a single shaded cell in the time plot 308. However, the preparation of femur (i.e., step 7) and preparation of tibia (i.e., step 9) events each include two shaded cells in their respective rows. This suggests that each of these two events is of a longer duration than the rest of the events (e.g., twice as long).

Additionally, the time plot 308 includes indicators at cells 314 and 316, which cells are associated with events expected to cause a significant physiological response (e.g., resulting in a spike in a biometric signal) in a patient. As discussed above, these types of events can cause physiological metrics to spike and cause a clinician and/or an infusion device to overcorrect in response.

Accordingly, in some implementations, providing the information contained in the timeline 300, discussed above, to the intelligent control module (“ICM”) 202 enables the ICM 202 to predict the knee incision and femoral component implantation events. The ICM 202 may then provide a bolus of medication (e.g., an anesthetic, an analgesic) to the patient prior to the events to prevent a spike in a physiological metric of the patient. Alternatively, the ICM 202 may prompt an operator of the infusion device to provide the bolus of medication to the patient. In either instance, the ICM 202 utilizes the information of the timeline 300 in order to improve the knee replacement surgery. One improvement includes the timely administration of a safe quantity of medication to increase patient comfort. A second improvement includes the gradual administration of medication over time to maintain a physiological metric of a patient within a target range which, as discussed, can conserve medication and avoid under or over administration.

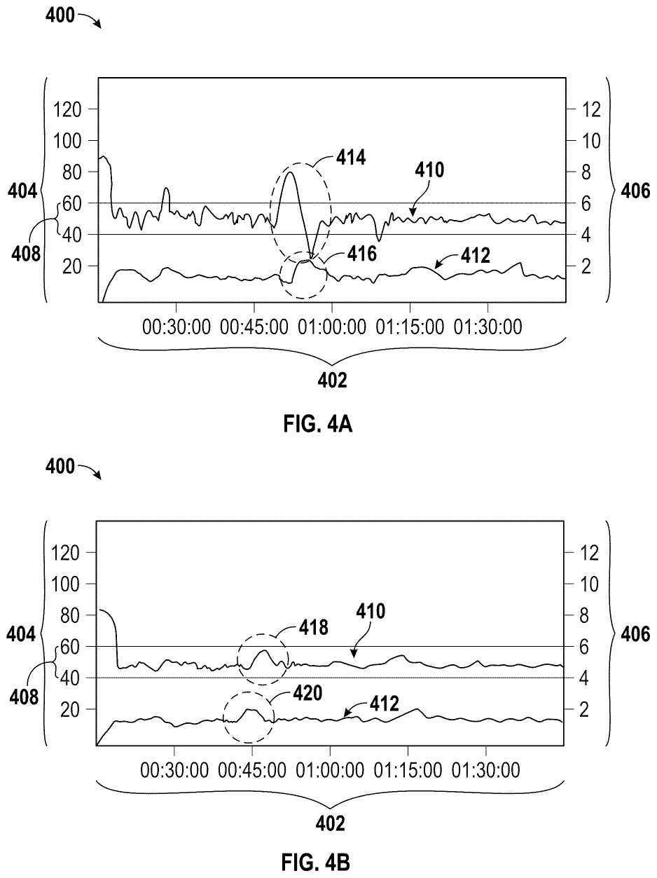

FIGS. 4A and 4B are example line plots 400 and 401 that each include a biometric sensor readout (see sensor line 410) and a medication infusion metric (see medication line 412), according to aspects of the subject technology. Each figure also includes an x-axis (see time axis 402) representing time, a left y-axis (see sensor axis 404) corresponding to the biometric sensor readout, and a right y-axis (see medication axis 406) corresponding to the medication infusion metric. Both figures also include a target zone 408 that represents an ideal range for the biometric sensor readout. For example, the target zone may correspond to an ideal level of anesthesia while a patient is under anesthesia during a surgery.

Referring now to FIG. 4A, real time measurements are received from a sensor 216 and represented as the sensor line 410 of the line plot 400. In the depicted example, a spike is measured at time 414. Medical procedures often include events that may cause a spike in a measured physiological metric of a patient. For example, a typical knee replacement surgery includes at least two events that may cause a physiological metric to spike: a knee incision (e.g., step 5 of timeline 300), and an implantation of a femoral component (e.g., step 8).

The physiological metric (e.g., measured by biometric sensors 216) associated with the sensor line 410 may include a BIS metric, an EMG metric, a blood glucose metric, and so on. For example, the intelligent control module (“ICM”) 202 may monitor a BIS level of a patient under anesthesia. Or the ICM 202 may monitor the blood glucose level of a patient being fed via an enteral pump.

A target zone 408 in which the sensor readings correspond to a therapeutic range is shown. In some implementations, when an anesthetic drug is being administered, the sensor 216 may include a BIS sensor and the target zone 408 may be a range of a measured value that corresponds to the patient being in a hypnotic state (e.g., under anesthesia). In some implementations, when the medication includes a vasopressor, the sensor 216 may include a biometric sensor configured to measure a cardiac output, and the target zone 408 may be a range of the cardiac output in which the cardiac output is medically stable.

According to various implementations, the ICM 202 monitors the sensor measurements and controls the infusion pump to maintain the measurements in the target zone 408. If a measurement drifts or spikes beyond the target zone 408, the ICM 202 administers an amount (e.g., an increased amount) of a medication (e.g., remifentanil, propofol, insulin) to cause the sensor line 410 to return to the target zone 408. In FIG. 4A, a temporary increase in the medication line 412 occurs at time 416, shortly after a spike is detected, to bring the spike back into the target zone 408.

According to various implementations, the ICM 202 is configured to control the pump to provide an amount (e.g., an increased amount) of the medication to the patient prior to a potential spike event associated with an adverse event (e.g., too much pain, exiting anesthesia prematurely), thereby avoiding the adverse event causing the spike and decreasing the height of any spike in the sensor line 410 resulting from the adverse event. Accordingly, in FIG. 4B, the pump is controlled to administer the medication before the spike is detected. In some implementations, the ICM 202 is configured to automatically detect, based on sensor data and the current step in the timeline 308, the likelihood of an upcoming spike and respond accordingly, whether by automatically initiating an infusion of the medication to the patient or by prompting an operator of an infusion pump to initiate the infusion of the medication.

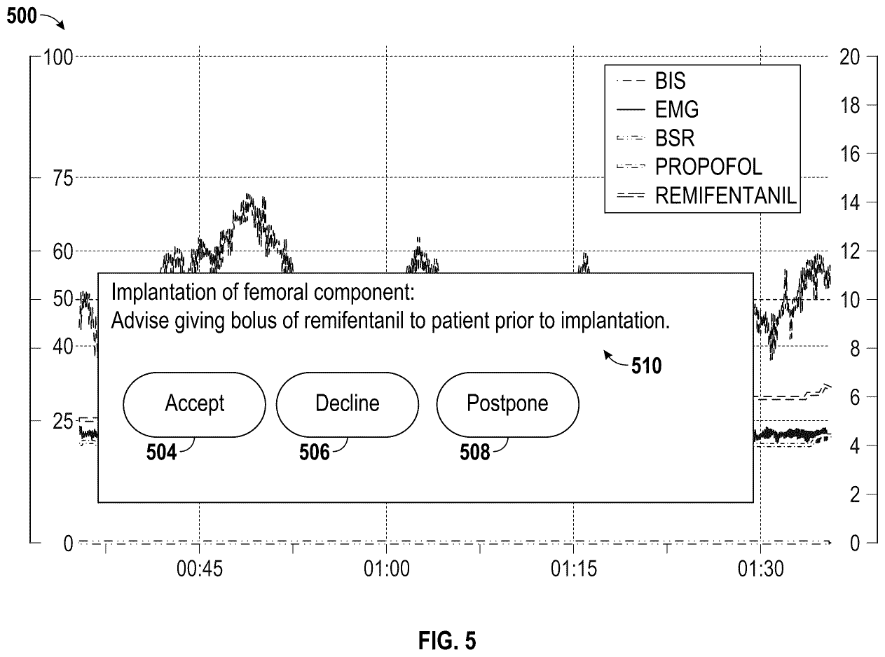

FIG. 5 is an example user interface 500 including a prompt 510 to initiate administration of a medication in anticipation of a physiological change for which the medication is prescribed, according to aspects of the subject technology. The example user interface 500 may be displayed on a display of the intelligent control module (“ICM”) 202, on an infusion device 214, or on an attached infusion module, pump (e.g., infusion module 116, or syringe pump(s) 220 or 222), or another electronic device specifically configured for infusion therapy (e.g., device terminal 132). As illustrated, the displayed prompt 510 indicates to an operator that an event (“Implantation of femoral component”) is about to occur. According to various implementations, some events in timeline 308 may be associated with a potential for physiologically generating a spike in a measured physiological metric of a patient. Accordingly, the prompt 510 advises providing an amount of a medication (e.g., remifentanil) to the patient prior to the occurrence of the event.

In some implementations, the user interface 500 also includes an “Accept” button 504, a “Decline” button 506, and/or a “Postpone” button 508. For example, selecting the “Accept” button 504 may cause the ICM 202 to initiate administration of the recommended medication to the patient via the infusion device, selecting the “Decline” button 506 may cause the display 500 to disappear, and selecting the “Postpone” button 508 may cause the display 500 to disappear for a predetermined amount of time, after which the display 500 may again appear. Accordingly, the operator may respond to the prompt 510 by selecting one of the “Accept” button 504, the “Decline” button 506, or the “Postpone” button 508. Additionally, or in the alternative, the operator may respond to the prompt 510 by selecting a button of an internet-of-things (IoT) device.

As illustrated, in some implementations, the prompt 510 includes an indication of an upcoming procedural event. For example, the prompt 510 indicates that the current procedure is advancing to a stage at which a femoral component will soon be implanted into a patient (e.g., as part of a knee replacement surgery). As discussed previously, the system may be programmed to associate femoral component implantation with a spike in a measured physiological metric of a patient undergoing a knee replacement surgery.

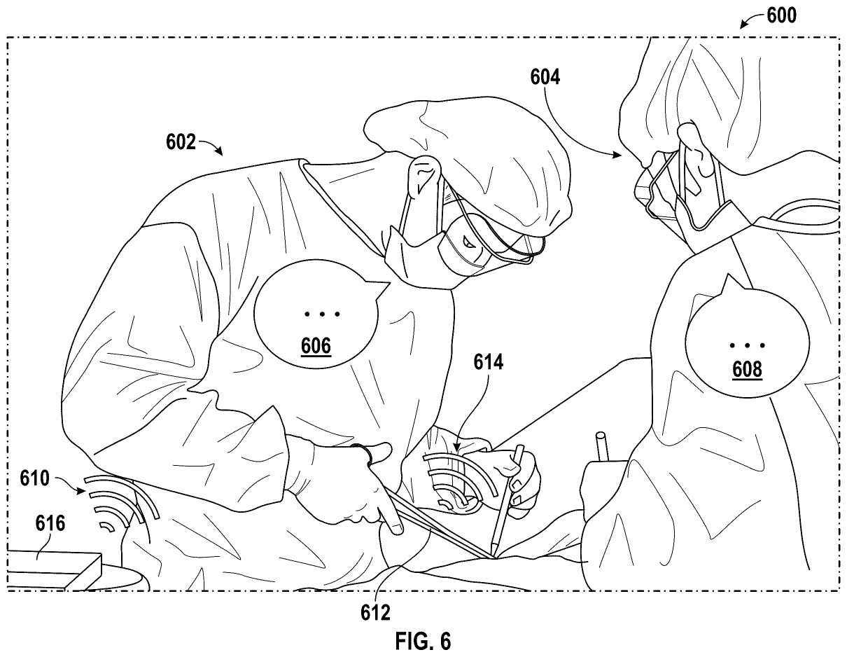

FIG. 6 depicts an example system 600 for detection of surgical events, according to aspects of the subject technology. As illustrated, a first surgeon 602 and a second surgeon 604 are performing a surgery on a patient. While performing the surgery, the surgeons 602 and 604 are speaking with each other, as indicated by speech bubbles 606 and 608. For example, the surgeons may be discussing the surgery, such as the procedure they are currently performing or an upcoming procedure.

In some implementations, the intelligent control module (“ICM”) 202 receives audio data that includes words spoken by the surgeons 602 and 604 (see speech bubbles 606 and 608). For example, if the surgeons are performing a knee replacement surgery, the spoken words may include keywords that indicate the current progress of the surgery. The words “scalpel,” or “incision” may indicate that the surgeons 602 and 604 are preparing to make an incision at the knee of the patient (see timeline 300, step 5. The words “prepare,” “femoral component,” “implant,” or “femur” may indicate that the surgeons are preparing to implant a femoral component into the patient's knee (see timeline 300, step 8).

Accordingly, in some implementations, the ICM 202 monitors audio and analyzes (e.g., using automatic speech recognition, natural language processing, and/or a machine learning model) the audio data to determine whether an event in a medical procedure is ongoing, upcoming, or completed. In this regard, as discussed in more detail below with respect to example process 800, the ICM 202 can determine that a procedural event expected to cause a spike in a physiological metric of a patient is about to occur, for example, based on the audio data (e.g., in conjunction with data regarding the events included in the surgery, such as the data illustrated in timeline 300).

Similarly, the ICM 202 can also use the audio data to determine what kind of medical procedure (e.g., a knee replacement surgery) is being performed. In some implementations, the ICM 202 uses the audio data to determine the type of medical procedure and then retrieves information regarding the medical procedure. For example, the control device may compare spoken words to keywords associated with different procedures and determine a respective procedure is being performed based on the comparison. The control device may then load the events included in the medical procedure and determine which of the events in the medical procedure are expected to cause a spike in a physiological metric of a patient.

The control device may further be configured to detect the use of instruments within a medical area such as an operating room. In the depicted example, medical devices and instruments (e.g., forceps 612, instrument tray 616, etc.) are configured with signal transmitters and/or receivers. In the depicted example, the forceps 612 and an instrument tray 616 each emit respective signals 610 and 614. For example, when the tray is brought into the room, a reader device may detect the signal being emitted from the tray. The ICM 202 may confirm that an event is being performed or is upcoming based on the tray being associated with the event. As another example, the instrument tray 616 may be configured with an RFID reader and instruments such as the depicted forceps 612 include RFID tags that allows the instrument(s) to be detected by the tray when the forceps 612 are in close proximity with the instrument tray 616 (e.g., laying in the instrument tray 616). Likewise, the instrument tray 616 may detect when the forceps 612 are not in close proximity with the instrument tray 616 (e.g., suggesting the forceps are being used as part of a medical procedure).

According to some implementations, the ICM 202 may be operably connected to one or more image sensing devices (e.g., cameras) configured to detect motion of objects within the medical area. The received image data may be processed using machine vision and, using image recognition, the control device may detect the use of objects associated with known events. The control device may further determine whether the object is in use and/or whether the event is completed based on motion of the detected object, also detected by machine vision. Together with location data provided by the previously described signal detection, and stored event data, the ICM 202 may determine that an event in a medical procedure is completed, ongoing, or upcoming.

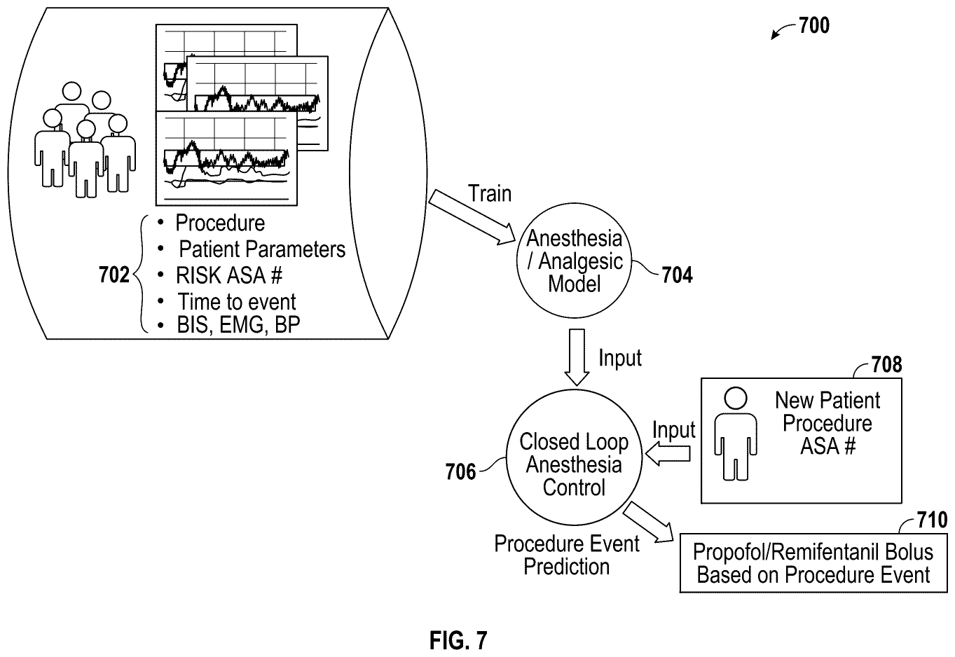

FIG. 7 illustrates a training flow 700 for a machine learning model 704 in conjunction with a closed-loop algorithm 706, according to aspects of the subject technology. According to various implementations, the intelligent control module (“ICM”) 202 implements a control algorithm (e.g., a closed-loop anesthesia control algorithm 706). In some implementations, the control algorithm(s) may include a machine learning algorithm. In some implementations, a machine learning model 704 provides input to the algorithm. In this manner, the machine learning model 704 may assist the ICM 202 in determining whether a procedural event is about to occur and whether a spike in a measured physiological parameter of a patient is about to occur in accordance with the procedural event.

The machine learning model 704 can be trained with data 702 that includes information regarding a medical procedure. For example, the data 702 may include a length of the medical procedure, information regarding the instruments (e.g., a scalpel, a pair of forceps, a drill) or equipment (e.g., a ventilator, an infusion pump, a monitor) required for the medical procedure, events (e.g., events 302) involved in the medical procedure (e.g., a knee replacement surgery, as illustrated in timeline 300), and/or any of the information discussed above with respect to timeline 300. The machine learning model 704 can also be trained with data 702 that includes information regarding a patient (e.g., a patient about to undergo the medical procedure, a patient that has undergone the medical procedure). For example, the data 702 may include a height, a weight, a body mass index (BMI), an HR, a blood pressure, a medical history, or a disease state of the patient. The data 702 may also include an American Society of Anesthesiologists (ASA) physical status (PS) classification number of a patient, which relates to the patient's pre-anesthesia medical co-morbidities.

Patient information may impact the length or sequencing of events in a medical procedure and therefore may be used in predicting whether a step in a medical procedure will occur. Further, patient information may affect the way the patient responds to a particular step in the medical procedure. According to various implementations, the patient information may provide data on which events are might cause a spike in a physiological parameter of the patient. Moreover, patient information may inform the ICM 202 as to the way the patient will respond to a particular medication (e.g., requiring that the medication is provided to the patient at an increased amount of time prior to a particular step in the medical procedure to avoid a spike in a physiological metric of the patient).

The machine learning model 704 may be further trained with sensor measurements (e.g., from a BIS sensor, an EMG sensor, a blood pressure sensor) during a medical procedure. The sensor measurements may be captured prior to, during, or after a particular step in the medical procedure. Accordingly, the sensor measurements may be used to train the machine learning model 704 to recognize whether a particular step in a medical procedure is about to occur, is ongoing, or was completed by recognizing sensor measurements similar to that of the training data. In some implementations, the data 702 includes sensor values captured prior, during, or after an administration of a medication to the patient.

In some implementations, the ICM 202 uses the input from the machine learning model 704 in determining whether a procedural event (e.g., a procedural event expected to cause a spike in a physiological metric of the patient) is about to occur. The machine learning model may be used by the ICM 202 in conjunction with information pertaining to a current patient 708 (e.g., a medical procedure prescribed for the current patient, the current patient's ASA PS number, the current patient's medical history) to determine whether the procedural event is about to occur, as well as to determine how to prevent a physiological spike from the procedural event (e.g., which medication to provide to the current patient, how much of the medication to provide, how early before the procedural event to provide the medication).

The input from the machine learning model 704 may improve the performance of the ICM 202 by informing the ICM 202 of how similar patients (e.g., patients with similar medical histories, weights, heights, etc.) responded to events in the medical procedure or to administration of a medication (e.g., prior to a step in the medical procedure). Thus, the ICM 202 can more appropriately respond to an upcoming procedural event by administering a medication to the current patient or by prompting an operator of the ICM 202 to initiate an infusion of the medication (see response 710).

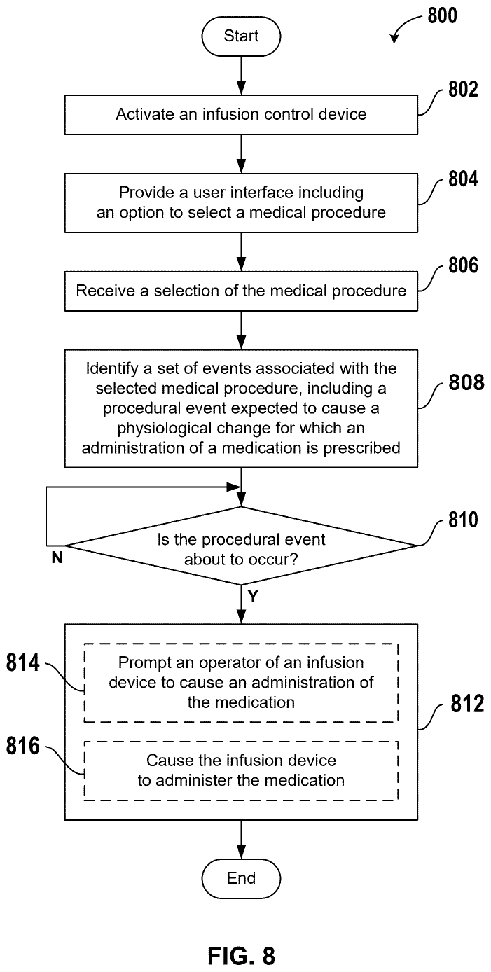

FIG. 8 depicts an example process 800 for intelligently determining when to initiate an infusion of a medication, according to aspects of the subject technology. One or more blocks of process 800 may be implemented, for example, by one or more computing devices, such as the PCD 112, the infusion module 116, the device terminal 132, the ICM 202, or other infusion device fusion devices/pumps. In some implementations, one or more of the blocks may be implemented based on one or more machine learning algorithms. In some implementations, one or more of the blocks may be implemented apart from other blocks, and by one or more different processors or devices. Further, for explanatory purposes, the blocks of example process 800 are described as occurring in serial, or linearly. However, multiple blocks of example process 800 may occur in parallel. Additionally, the blocks of example process 800 need not be performed in the order shown and one or more of the blocks of example process 800 need not be performed.

In the depicted example process 800, an intelligent control module is provided (802). The intelligent control module may be implemented by, for example, the intelligent control module (“ICM”) 202, or a processor associated with a medical device such as a PCD 112. The intelligent control module may include a control interface configured to exchange infusion information with an infusion device (including, e.g., an infusion module or control module) and to receive physiological information from a patient sensor (e.g., biometric sensors 216). As discussed herein, the intelligent control module may be connected to the infusion device or patient sensor wirelessly or via an electrical connection. Alternatively, the infusion device or patient sensor may be subcomponents of the intelligent control module.

A user interface (e.g., user interface device 154, user interface 204) provides an option to select a medical procedure (804). In some implementations, the user interface is provided via a display, such as a display of the intelligent control module (e.g., user interface 204) or a display of the infusion device. In some implementations, the user interface also includes options to select parameters associated with the medical procedure (e.g., a duration, a type, a scheduled start time) or parameters associated with controlling an infusion device to administer a medication (e.g., medication type, flow rate, volume, etc.).

A selection of the medical procedure (e.g., a selection of a knee replacement surgery) is received via the user interface (806). The selection of the medical procedure may be received, for example, via input made at the user interface, or it may be received from an electronic medical records (EMR) system over a network. Additionally, or in the alternative, the selection of the medical procedure may be received via input received at an electronic device (e.g., a mobile device) connected to the intelligent control module.

The process 800 continues by identifying (808) a set of events (e.g., events 302) associated with the selected medical procedure. In this regard, the set of events may include a procedural event (e.g., timeline 300, “Incision at knee”) expected to cause a physiological change for which an administration of a medication (e.g., propofol, remifentanil, insulin) is prescribed. Training data may associate a knee incision event in a knee replacement surgery with a spike in measured BIS or EMG values. Administration of propofol or remifentanil may be prescribed for the knee incision event to prevent or alleviate the spike in the patient's BIS or EMG values.

In some implementations, expected physiological changes may be based on physiological data associated with the procedural event exiting a target zone. For example, a knee replacement surgery may call for keeping a BIS value of a patient within a target zone while the patient is under anesthesia, and a knee incision event and an implantation of a femoral component event (see timeline 300) may be expected to cause the BIS value of the patient to exit the target zone. For example, a target zone for the BIS value may be between 40 and 60.

As described previously, a timeline 300 associated with the selected medical procedure may be retrieved from the external database 137 in connection with identifying the set of events for the selected medical procedure. For example, if the selected medical procedure is a knee replacement surgery, the data may include step numbers (e.g., step numbers 304) and descriptions (e.g., descriptions 306) associated with each respective event of the knee replacement surgery. Additionally, the data may include chronological information regarding the events involved in the surgery (see, e.g., timeline 300). Further, the data may include information regarding which of the events is likely to cause a significant change in a physiological metric of a patient.

Furthermore, the data may include information regarding the instruments required (e.g., forceps, a scalpel, a drill) for the medical procedure, as well as which events the instruments are associated with. Also, the data may include language information regarding the words used by medical professionals while performing the medical procedure. For example, the language information may be used to assist in determining when a procedural event will occur.

In some implementations, administering the medication to the patient (e.g., via infusion module 116, infusion device 214) is prescribed for returning the physiological metric of the patient to the target zone after an occurrence of the procedural event. For example, administering propofol or remifentanil may be prescribed for returning the BIS value of the patient to the target zone (e.g., a target anesthesia zone) after the occurrence of the knee incision or femoral component implantation events.

The process 800 continuously monitors the event data and/or sensor data to determine whether the procedural event is about to occur (810). In some implementations, determining (810) whether the procedural event is about to occur includes receiving (e.g., from a microphone) audio data captured during the medical procedure. For example, the audio data may include speech data (e.g., human speech data) that describes the selected medical procedure. Further, determining (810) whether the procedural event is about to occur may be performed based on the selected medical procedure (e.g., a knee replacement surgery), the procedural event (e.g., a knee incision), and the audio data. For an example implementation of this, see FIG. 6.

One or more of the events identified and/or received in step 808 may each be associated with a predetermined start time, for example, relative to a procedure start time. For example, the procedural event monitored in step 810 may be expected to occur at an amount of time (e.g., forty minutes) after the procedure start time. In this regard, the intelligent control module determining that the procedural event is about to occur may include determining that a predetermined amount of time has passed since the procedure start time or a predetermined amount of time has passed from the start or completion of a preceding event. For example, the intelligent control module may determine that a knee incision event (e.g., timeline 300, step 5) is about to occur based on determining that an induction event (e.g., timeline 300, step 1) began at twenty minutes ago (e.g., at 2:30 PM), and determining that twenty minutes (e.g., a predetermined portion of the amount of time) have passed since the induction event began.

Furthermore, in some implementations, determining that the detectable event began at the predetermined time includes determining that physiological information satisfies a predetermined threshold. For example, determining that a spinal nerve block event (see timeline 300, “Spinal nerve block”) began may include determining that a BIS, EMG, or HR level of a patient satisfies (e.g., is less than) a respective BIS, EMG, or HR threshold.

In some implementations, determining whether the procedural event is about to occur includes receiving (e.g., from a camera, a 60 GHz radar) video data captured during the medical procedure. For example, the video data may capture a surgeon performing a surgery. The video data may be provided to a vision and position detection algorithm capable of determining, for example, that an event in the medical procedure is completed, ongoing, or upcoming, or that an instrument or piece of equipment required for the medical procedure is in-use or out-of-use.

In some implementations, determining whether the procedural event is about to occur includes receiving an input from an IoT button or trigger device. The input may be from a medical professional indicating that the procedural event is about to occur. For example, in a knee replacement surgery, a surgeon may click a button on an IoT device to indicate to an anesthesiologist that the surgeon will soon perform a knee incision event (e.g., in ten minutes).

After determining that the procedural event is about to occur (810-Y), the process 800 includes performing (812) one or more of the following: (a) prompting (e.g., prompt 510) an operator of an infusion device to cause an administration of the medication (814); or (b) causing an infusion device to administer the medication (816).

In some implementations, the process 800 includes prompting the operator to cause an adjustment of the infusion device to administer the medication at a second rate different from a first rate (e.g., at which the medication was being administered). Further, in some implementations, the process 800 includes causing the adjustment of the infusion device to administer the medication at the second rate. For example, the adjustment may include increasing a flow rate of the medication (i.e., the second rate is greater than the first rate). If the procedural event is expected to cause a positive change in the physiological metric (e.g., an upward spike in a pain-related metric), then the flow rate of the medication (e.g., an analgesic drug) may be increased to prevent the positive change. On the other hand, if the procedural event is expected to cause a negative change in the physiological metric (e.g., a downward spike in a BIS metric), then the flow rate of the medication (e.g., an anesthetic drug) may be decreased, for example, to prevent the negative change.

In some implementations, prompting the operator of the infusion device to cause the administration of the medication includes prompting the operator of the infusion device to adjust an amount of a medication that is currently being administered to a patient. For example, if the patient is being administered propofol at a rate of 4 mg/kg/hr, the intelligent control module may prompt the operator to administer propofol to the patient at a rate of 6 mg/kg/hr (e.g., for five minutes, indefinitely). Likewise, in some implementations, causing the administration of the medication via the infusion device includes adjusting an amount of a medication that is currently being administered to a patient.

In some implementations, the selected medical procedure includes a surgery, and the procedural event expected to cause a physiological change in the patient includes a step in the surgery. Further, the medication prescribed for the procedural event is an analgesic, an anesthetic, or an antihypotensive agent. Moreover, causing the infusion device to administer the medication prior to the occurrence of the procedural event includes causing the infusion device (e.g., via the control interface) to administer the analgesic, the anesthetic, or the antihypotensive agent prior to the step in the surgery.

Further, in some implementations, the control interface may be configured to receive an instrument status from an instrument sensor (see, e.g., signal indicators 614 and 610). For example, the instrument status may indicate whether the status of an instrument (e.g., a scalpel, a pair of forceps) is an in-use status or a not-in-use status (e.g., whether the instrument is being used). Moreover, the selected medical procedure may require a predetermined change in the instrument status (e.g., from the in-use status to the not-in-use status, vice versa) prior to the occurrence of the procedural event. Additionally, determining that the procedural event is about to occur may include detecting (e.g., via the control interface) the predetermined change in the instrument status.

According to some implementations, the instrument includes an RFID tag, and detecting the change in the instrument status includes detecting a change in an RFID signal from the RFID tag. For example, as described previously with respect to FIG. 6, in some implementations, an instrument tray includes an RFID reader configured to detect when the RFID tag of the instrument is in close proximity of the instrument tray. Accordingly, detecting the change in the RFID signal from the RFID tag may include detecting that the instrument is no longer in close proximity of the instrument tray, or that it moved within close proximity of the instrument tray.

In some implementations, the control interface is further configured to exchange nutritional information with a medical device (e.g., infusion module 116, infusion device 214, an enteral pump). Additionally, the selected medical procedure includes an administration of nutrition to a patient via the medical device. Further, the medication includes insulin. And, causing the infusion device to administer (e.g., automatically) the medication prior to the occurrence of the procedural event includes causing the infusion device to administer the insulin prior to the administration of the nutrition to the patient via the medical device.

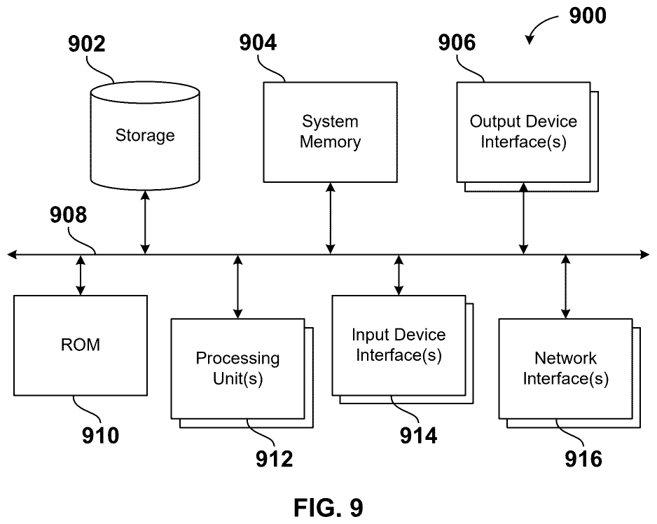

FIG. 9 is a conceptual diagram illustrating an example electronic system 900 for intelligently determining when to initiate an infusion of a medication, according to aspects of the subject technology. Electronic system 900 may be implemented by a computing device for execution of software associated with portions or steps of process 800, or components and methods provided by FIGS. 1-7. In this regard, electronic system 900 may include the PCD 112, the infusion module 116, the device terminal 132, the ICM 202, or the infusion device 214. The electronic system 900 may also include a specifically configured personal computer or a mobile device for infusion such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 900 may also include various types of computer readable media and interfaces for various other types of computer readable media. In the depicted example, electronic system 900 includes a bus 908, a processing unit(s) 912, a system memory 904, a read-only memory (ROM) 910, a permanent storage device 902, an input device interface(s) 914, an output device interface(s) 906, and a network interface(s) 916. In some implementations, electronic system 900 may include or be integrated with other computing devices or circuitry for operation of the various components and methods previously described.

Bus 908 collectively represents all system, peripheral, and chipset buses that communicatively connect the numerous internal devices of electronic system 900. For instance, bus 908 communicatively connects processing unit(s) 912 with ROM 910, system memory 904, and permanent storage device 902.

From these various memory units, processing unit(s) 912 retrieves instructions to execute and data to process, in order to execute the processes of the subject disclosure. The processing unit(s) 912 can be a single processor or a multi-core processor in different implementations.

ROM 910 stores static data and instructions that are needed by processing unit(s) 912 and other modules of the electronic system. Permanent storage device 902, on the other hand, is a read-and-write memory device. This device is a non-volatile memory unit that stores instructions and data even when electronic system 900 is powered off. Some implementations of the subject disclosure use a mass-storage device (such as a magnetic or optical disk and its corresponding disk drive) as the permanent storage device 902.

Other implementations use a removable storage device (such as a floppy disk, flash drive, and its corresponding disk drive) as permanent storage device 902. Like permanent storage device 902, system memory 904 is a read-and-write memory device. However, unlike storage device 902, system memory 904 is a volatile read-and-write memory, such as random-access memory (RAM). System memory 904 stores some of the instructions and data that the processor needs at runtime. In some implementations, the processes of the subject disclosure are stored in system memory 904, permanent storage device 902, and/or ROM 910. From these various memory units, the processing unit(s) 912 retrieves instructions to execute and data to process, in order to execute the processes of some implementations.

Bus 908 also connects to input device interface(s) 914 and output device interface(s) 906. Input device interface(s) 914 enable the user to communicate information and select commands to the electronic system. Input devices used with input device interface(s) 914 include, for example, alphanumeric keyboards and pointing devices (also called “cursor control devices”). Output device interface(s) 906 enable, for example, the display of images generated by the electronic system 900. Output devices used with output device interface(s) 906 include, for example, printers and display devices, such as cathode ray tubes (CRT) or liquid crystal displays (LCD). Some implementations include devices such as touchscreens that functions as both input and output devices.

Furthermore, bus 908 also couples electronic system 900 to a network (not shown) through network interface(s) 916. Network interface(s) 916 may include, for example, a wireless access point (e.g., Bluetooth or Wi-Fi) or radio circuitry for connecting to a wireless access point. Network interface(s) 916 may also include hardware (e.g., ethernet hardware) for connecting the computer to a part of a network of computers such as a local area network (LAN), a wide area network (WAN), wireless LAN, an intranet, or a network of networks, such as the Internet. Any or all components of electronic system 900 can be used in conjunction with the subject disclosure when specifically configured with one of more of the features described.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field-programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices specifically configured with one or more of the features described above. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. For example, feedback provided to the user can be any form of sensory feedback (e.g., visual feedback, auditory feedback, tactile feedback), and input from the user can be received in forms such as acoustic, speech, gesture, or tactile input. In addition, a computer can interact with a user by sending documents to and receiving documents from a device that is used by the user (e.g., by sending web pages to a web browser on a user's client device in response to requests received from the web browser).

Implementations of the subject matter described in this specification can be implemented in a specifically configured computing system that includes a back end component (e.g., a data server), or that includes a specifically configured middleware component (e.g., an application server), or that includes a specifically configured front end component (e.g., a client computer having a graphical user interface or a Web browser through which a user can interact with an implementation of the subject matter described in this specification), or any combination of one or more such back end, middleware, or front end components. The components of the system can be interconnected by one or more forms or mediums of digital data communication, such as a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include specifically configured clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art will appreciate that the various illustrative blocks, modules, elements, components, methods, and algorithms described herein may be implemented as electronic hardware, computer software, or a combination thereof. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. An intelligent control device for intelligent infusion, comprising: a control interface configured to exchange infusion information with an infusion device and physiological information with a patient sensor; a processor; and a non-transitory, computer-readable storage medium storing instructions that, when executed by the processor, cause the intelligent control device to: provide, via a display device, a user interface comprising an option to select a medical procedure; receive, via the user interface, a selection of the medical procedure; operate, via the control interface, the infusion device to administer a medication at a first rate; identify a set of events associated with the selected medical procedure, wherein the set of events comprises a procedural event that is expected to cause a physiological change for which an administration of the medication is prescribed; determine that the procedural event is about to occur based on the infusion information, the physiological information, or the selected medical procedure; and responsive to determining that the procedural event is about to occur, and prior to an occurrence of the procedural event: prompt, via the display device, an operator of the infusion device to cause an adjustment of the infusion device to administer the medication at a second rate different from the first rate; or cause, via the control interface, the adjustment of the infusion device to administer the medication at the second rate.
Clause 2. The intelligent control device of Clause 1, wherein: the physiological change expected to be caused by the procedural event comprises a physiological metric of a patient exiting a target zone; and administering the medication to the patient, via the infusion device and after the occurrence of the procedural event, is prescribed for returning the physiological metric of the patient to the target zone.
Clause 3. The intelligent control device of any one of Clauses 1 through 2, wherein causing the administration of the medication via the infusion device comprises adjusting an amount of a medication currently being administered to a patient.
Clause 4. The intelligent control device of any one of Clauses 1 through 3, wherein: the control interface is further configured to receive audio data from a microphone; and determining that the procedural event is about to occur comprises: receiving, from the microphone, audio data captured in a predetermined space associated with the selected medical procedure and during a predetermined time associated with the selected medical procedure, wherein the audio data includes human speech data corresponding to the selected medical procedure; and determining, based on the selected medical procedure, the procedural event, and the human speech data, that the procedural event is about to occur.
Clause 5. The intelligent control device of any one of Clauses 1 through 4, wherein: the set of events associated with the selected medical procedure comprises a detectable event that is to begin at a predetermined time; the procedural event is to occur at an amount of time after the predetermined time; and determining that the procedural event is about to occur comprises: detecting that the detectable event began at the predetermined time; and determining that a predetermined portion of the amount of time has passed since the predetermined time.
Clause 6. The intelligent control device of Clause 5, wherein determining that the detectable event began at the predetermined time comprises determining that the physiological information satisfies a predetermined threshold.
Clause 7. The intelligent control device of any one of Clauses 1 through 6, wherein: the selected medical procedure comprises a surgery; the procedural event comprises a step in the surgery; the medication comprises an analgesic, an anesthetic, or an antihypotensive agent; and causing the infusion device to administer the medication prior to the occurrence of the procedural event comprises causing the infusion device to administer the analgesic, the anesthetic, or the antihypotensive agent prior to the step in the surgery.
Clause 8. The intelligent control device of Clause 7, wherein: the control interface is further configured to receive an instrument status from an instrument sensor, wherein the instrument status indicates whether an instrument is in at least one of an in-use status or a not-in-use status; the selected medical procedure requires that the instrument status change from the not-in-use status to the in-use status prior to the occurrence of the procedural event; and determining that the procedural event is about to occur comprises detecting, via the control interface, that the instrument status changed from the not-in-use status to the in-use status.
Clause 9. The intelligent control device of Clause 8, wherein: the instrument comprises a radio-frequency identification (RFID) tag; and detecting that the instrument status changed comprises detecting a change in an RFID signal from the RFID tag.
Clause 10. The intelligent control device of any one of Clauses 1 through 6, wherein: the control interface is further configured to exchange nutritional information with a medical device that is configured to administer nutrition to a patient; the procedural event comprises an administration of the nutrition to the patient via the medical device; the medication comprises insulin; and causing the infusion device to administer the medication prior to the occurrence of the procedural event comprises causing the infusion device to administer the insulin prior to the administration of the nutrition to the patient via the medical device.
Clause 11. A computer-implemented method of intelligent infusion, comprising:
activating an intelligent control device comprising a control interface configured to exchange infusion information with an infusion device and physiological information with a patient sensor; providing, via a display device, a user interface comprising an option to select a medical procedure; receiving, via the user interface, a selection of the medical procedure; operating the infusion device, via the control interface, to administer a medication at a first rate; identifying a set of events associated with the selected medical procedure, wherein the set of events comprises a procedural event that is expected to cause a physiological change for which an administration of the medication is prescribed; determining that the procedural event is about to occur based on the infusion information, the physiological information, or the selected medical procedure; and responsive to determining that the procedural event is about to occur, and prior to an occurrence of the procedural event: prompting, via the display device, an operator of the infusion device to cause an adjustment of the infusion device to administer the medication at a second rate different from the first rate; or causing, via the control interface, the adjustment of the infusion device to administer the medication at the second rate.
Clause 12. The computer-implemented method of Clause 11, wherein: the physiological change expected to be caused by the procedural event comprises a physiological metric of a patient exiting a target zone; and administering the medication to the patient, via the infusion device and after the occurrence of the procedural event, is prescribed for returning the physiological metric of the patient to the target zone.
Clause 13. The computer-implemented method of any one of Clauses 11 through 12, wherein causing the administration of the medication via the infusion device comprises adjusting an amount of a medication currently being administered to a patient.
Clause 14. The computer-implemented method of any one of Clauses 11 through 13, wherein: the control interface is further configured to receive audio data from a microphone; and determining that the procedural event is about to occur comprises: receiving, from the microphone, audio data captured in a predetermined space associated with the selected medical procedure and during a predetermined time associated with the selected medical procedure, wherein the audio data includes human speech data corresponding to the selected medical procedure; and determining, based on the selected medical procedure, the procedural event, and the human speech data, that the procedural event is about to occur.
Clause 15. The computer-implemented method of any one of Clauses 11 through 14, wherein: the set of events associated with the selected medical procedure comprises a detectable event that is to begin at a predetermined time; the procedural event is to occur at an amount of time after the predetermined time; and determining that the procedural event is about to occur comprises: detecting that the detectable event began at the predetermined time; and determining that a predetermined portion of the amount of time has passed since the predetermined time.
Clause 16. The computer-implemented method of Clause 15, wherein determining that the detectable event began at the predetermined time comprises determining that the physiological information satisfies a predetermined threshold.
Clause 17. The computer-implemented method of any one of Clause 11 through 16, wherein: the selected medical procedure comprises a surgery; the procedural event comprises a step in the surgery; the medication comprises an analgesic, an anesthetic, or an antihypotensive agent; and causing the infusion device to administer the medication prior to the occurrence of the procedural event comprises causing the infusion device to administer the analgesic, the anesthetic, or the antihypotensive agent prior to the step in the surgery.
Clause 18. The computer-implemented method of Clause 17, wherein: the control interface is further configured to receive an instrument status from an instrument sensor, wherein the instrument status indicates whether an instrument is in at least one of an in-use status or a not-in-use status; the selected medical procedure requires that the instrument status change from the not-in-use status to the in-use status prior to the occurrence of the procedural event; and determining that the procedural event is about to occur comprises detecting, via the control interface, that the instrument status changed from the not-in-use status to the in-use status.
Clause 19. The computer-implemented method of Clause 18, wherein: the instrument comprises a radio-frequency identification (RFID) tag; and detecting that the instrument status changed comprises detecting a change in an RFID signal from the RFID tag.
Clause 20. The computer-implemented method of any one of Clauses 11 through 16, wherein: the control interface is further configured to exchange nutritional information with a medical device that is configured to administer nutrition to a patient; the procedural event comprises an administration of the nutrition to the patient via the medical device; the medication comprises insulin; and causing the infusion device to administer the medication prior to the occurrence of the procedural event comprises causing the infusion device to administer the insulin prior to the administration of the nutrition to the patient via the medical device.

### FURTHER CONSIDERATION

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects.

Thus, the claims are not intended to be limited to the aspects shown herein but are to be accorded the full scope consistent with the language of the claims. For example, reference to an element in the singular is not intended to mean “one and only one” unless specifically so stated, but rather “one or more.” Moreover, unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to,” “operable to,” and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term “automatic,” as used herein, may include performance by a computer or machine without user intervention, for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface, or a UI) may refer to a network-based interface including data fields or other control elements for receiving input signals or providing electronic information or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, . NETT™, C, C++, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database, or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine-readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fu5y logic, pattern matching, a machine learning assessment model, or combinations thereof.

In some implementations, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. An intelligent control device for intelligent infusion, comprising:
a control interface configured to exchange infusion information with an infusion device and physiological information with a patient sensor;
a processor; and
a non-transitory, computer-readable storage medium storing instructions that, when executed by the processor, cause the intelligent control device to:
provide, via a display device, a user interface comprising an option to select a medical procedure;
receive, via the user interface, a selection of the medical procedure;
operate, via the control interface, the infusion device to administer a medication at a first rate;
identify a set of events associated with the selected medical procedure, wherein the set of events comprises a procedural event that is expected to cause a physiological change for which an administration of the medication is prescribed;
determine that the procedural event is about to occur based on the infusion information, the physiological information, or the selected medical procedure; and
responsive to determining that the procedural event is about to occur, and prior to an occurrence of the procedural event:
prompt, via the display device, an operator of the infusion device to cause an adjustment of the infusion device to administer the medication at a second rate different from the first rate; or
cause, via the control interface, the adjustment of the infusion device to administer the medication at the second rate.

2. The intelligent control device of claim 1, wherein:
the physiological change expected to be caused by the procedural event comprises a physiological metric of a patient exiting a target zone; and
administering the medication to the patient, via the infusion device and after the occurrence of the procedural event, is prescribed for returning the physiological metric of the patient to the target zone.

3. The intelligent control device of claim 1, wherein causing the administration of the medication via the infusion device comprises adjusting an amount of a medication currently being administered to a patient.

4. The intelligent control device claim 1, wherein:
the control interface is further configured to receive audio data from a microphone; and
determining that the procedural event is about to occur comprises:
receiving, from the microphone, audio data captured in a predetermined space associated with the selected medical procedure and during a predetermined time associated with the selected medical procedure, wherein the audio data includes human speech data corresponding to the selected medical procedure; and
determining, based on the selected medical procedure, the procedural event, and the human speech data, that the procedural event is about to occur.

5. The intelligent control device of claim 1, wherein:
the set of events associated with the selected medical procedure comprises a detectable event that is to begin at a predetermined time;
the procedural event is to occur at an amount of time after the predetermined time; and
determining that the procedural event is about to occur comprises:
detecting that the detectable event began at the predetermined time; and
determining that a predetermined portion of the amount of time has passed since the predetermined time.

6. The intelligent control device of claim 5, wherein determining that the detectable event began at the predetermined time comprises determining that the physiological information satisfies a predetermined threshold.

7. The intelligent control device of claim 1, wherein:
the selected medical procedure comprises a surgery;
the procedural event comprises a step in the surgery;
the medication comprises an analgesic, an anesthetic, or an antihypotensive agent; and
causing the infusion device to administer the medication prior to the occurrence of the procedural event comprises causing the infusion device to administer the analgesic, the anesthetic, or the antihypotensive agent prior to the step in the surgery.

8. The intelligent control device of claim 7, wherein:
the control interface is further configured to receive an instrument status from an instrument sensor, wherein the instrument status indicates whether an instrument is in at least one of an in-use status or a not-in-use status;
the selected medical procedure requires that the instrument status change from the not-in-use status to the in-use status prior to the occurrence of the procedural event; and
determining that the procedural event is about to occur comprises detecting, via the control interface, that the instrument status changed from the not-in-use status to the in-use status.

9. The intelligent control device of claim 8, wherein:
the instrument comprises a radio-frequency identification (RFID) tag; and
detecting that the instrument status changed comprises detecting a change in an RFID signal from the RFID tag.

10. The intelligent control device of claim 1, wherein:
the control interface is further configured to exchange nutritional information with a medical device that is configured to administer nutrition to a patient;
the procedural event comprises an administration of the nutrition to the patient via the medical device;
the medication comprises insulin; and
causing the infusion device to administer the medication prior to the occurrence of the procedural event comprises causing the infusion device to administer the insulin prior to the administration of the nutrition to the patient via the medical device.

11. A computer-implemented method of intelligent infusion, comprising:
activating an intelligent control device comprising a control interface configured to exchange infusion information with an infusion device and physiological information with a patient sensor;
providing, via a display device, a user interface comprising an option to select a medical procedure;
receiving, via the user interface, a selection of the medical procedure;
operating, via the control interface, the infusion device to administer a medication at a first rate;
identifying a set of events associated with the selected medical procedure, wherein the set of events comprises a procedural event that is expected to cause a physiological change for which an administration of the medication is prescribed;
determining that the procedural event is about to occur based on the infusion information, the physiological information, or the selected medical procedure; and
responsive to determining that the procedural event is about to occur, and prior to an occurrence of the procedural event:
prompting, via the display device, an operator of the infusion device to cause an adjustment of the infusion device to administer the medication at a second rate different from the first rate; or
causing, via the control interface, the adjustment of the infusion device to administer the medication at the second rate.

12. The computer-implemented method of claim 11, wherein:
the physiological change expected to be caused by the procedural event comprises a physiological metric of a patient exiting a target zone; and
administering the medication to the patient, via the infusion device and after the occurrence of the procedural event, is prescribed for returning the physiological metric of the patient to the target zone.

13. The computer-implemented method of claim 11-or wherein causing the administration of the medication via the infusion device comprises adjusting an amount of a medication currently being administered to a patient.

14. The computer-implemented method of claim 11, wherein:
the control interface is further configured to receive audio data from a microphone; and
determining that the procedural event is about to occur comprises:
receiving, from the microphone, audio data captured in a predetermined space associated with the selected medical procedure and during a predetermined time associated with the selected medical procedure, wherein the audio data includes human speech data corresponding to the selected medical procedure; and
determining, based on the selected medical procedure, the procedural event, and the human speech data, that the procedural event is about to occur.

15. The computer-implemented method of claim 11, wherein:
the set of events associated with the selected medical procedure comprises a detectable event that is to begin at a predetermined time;
the procedural event is to occur at an amount of time after the predetermined time; and
determining that the procedural event is about to occur comprises:
detecting that the detectable event began at the predetermined time; and
determining that a predetermined portion of the amount of time has passed since the predetermined time.

16. The computer-implemented method of claim 15, wherein determining that the detectable event began at the predetermined time comprises determining that the physiological information satisfies a predetermined threshold.

17. The computer-implemented method of claim 11, wherein:
the selected medical procedure comprises a surgery;
the procedural event comprises a step in the surgery;
the medication comprises an analgesic, an anesthetic, or an antihypotensive agent; and
causing the infusion device to administer the medication prior to the occurrence of the procedural event comprises causing the infusion device to administer the analgesic, the anesthetic, or the antihypotensive agent prior to the step in the surgery.

18. The computer-implemented method of claim 17, wherein:
the control interface is further configured to receive an instrument status from an instrument sensor, wherein the instrument status indicates whether an instrument is in at least one of an in-use status or a not-in-use status;
the selected medical procedure requires that the instrument status change from the not-in-use status to the in-use status prior to the occurrence of the procedural event; and
determining that the procedural event is about to occur comprises detecting, via the control interface, that the instrument status changed from the not-in-use status to the in-use status.

19. The computer-implemented method of claim 18, wherein:
the instrument comprises a radio-frequency identification (RFID) tag; and
detecting that the instrument status changed comprises detecting a change in an RFID signal from the RFID tag.

20. The computer-implemented method of claim 11, wherein:
the control interface is further configured to exchange nutritional information with a medical device that is configured to administer nutrition to a patient;
the procedural event comprises an administration of the nutrition to the patient via the medical device;
the medication comprises insulin; and
causing the infusion device to administer the medication prior to the occurrence of the procedural event comprises causing the infusion device to administer the insulin prior to the administration of the nutrition to the patient via the medical device.
